(19) United States
(12) Patent Application Publication
(10) Pub. No.: US 2025/0017885 A1
(43) Pub. Date: Jan. 16, 2025

(54) TREATMENT OF INCLUSION BODY MYOSITIS WITH PHENYLBUTYRATE AND MONITORING OF TREATMENT

(71) Applicant: University of Kansas, Lawrence, KS (US)

(72) Inventors: Duaa Jabari, Overland Park, KS (US); Richard J. Barohn, Kansas City, MO (US); Mazen M. Dimachkie, Overland Park, KS (US); Heather Wilkins, Kansas City, KS (US)

(21) Appl. No.: 18/770,447

(22) Filed: Jul. 11, 2024

Related U.S. Application Data
(60) Provisional application No. 63/513,212, filed on Jul. 12, 2023.

(51) Int. Cl. A61K 31/192; C12Q 1/6876; G01N 33/52

(52) U.S. Cl. CPC A61K 31/192; C12Q 1/6876; G01N 33/52; G01N 2800/52

(57) ABSTRACT

A method for treating inclusion body myositis (IBM) in a subject can include: providing a subject that has IBM; administering phenylbutyrate (PBA) to the subject in a therapeutically effective amount to provide a treatment for the IBM; obtaining at least one biological sample from the subject after initiation of the treatment for the IBM; measuring a biological indicator in the at least one biological sample, wherein the biological indicator is mitochondrial membrane potential; and monitoring a change in the biological indicator as an indication of effectiveness of the treatment.

### CROSS-REFERENCE TO RELATED APPLICATIONS

This patent application claims priority to U.S. Provisional Application No. 63/513,212 filed Jul. 12, 2023, which provisional is incorporated herein by specific reference in its entirety.

### BACKGROUND

### Field

The present disclosure relates to compounds and/or materials for use as treatment of body myositis and monitoring the treatment.

### Description of Related Art

Inclusion body myositis (IBM) is an inflammatory muscle disease that occurs in older adults. The IBM disease state presents with slow progressive weakness and wasting of both proximal muscles (e.g., located on or close to the torso) and distal muscles (e.g., in or close to hands or feet). Typically, IBM is first apparent in the finger flexors and knee extensors. In IBM, two processes appear to occur in the muscles in parallel, one inflammatory and the other degenerative. In some instances, inflammation is evident from the invasion of muscle fibers by immune cells. Also, degeneration is characterized by the appearance of vacuoles (holes), deposits of abnormal proteins, and filamentous inclusions in the muscle fibers. However, there is currently no approved treatment for IBM.

U.S. Publication No. 2014/0206763A1 discloses a method of treating sporadic inclusion body myositis (s-IBM) or ameliorating one or more pathological phenotypes of s-IBM by administering to a patient in need thereof an effective amount of PBA.

Previously, PBA was used in a study to determine safety and tolerance of PBA in treating s-IBM (classic.clinicaltrials.gov/ct2/show/NCT04421677), which used 3 mg twice a day for 3 months.

The s-IBM is a severe, progressive muscle disease for which there is no enduring treatment (Nogalska A et al., Sodium phenylbutyrate reverses lysosomal dysfunction and decreases amyloid-β42 in an in vitro-model of inclusion-body myositis, Neurobiology of Disease, Volume 65, 2014, Pages 93-101, ISSN 0969-9961, doi.org/10.1016/j.nbd.2014.01.012). Pathologically characteristic are vacuolated muscle fibers having: accumulations of multi-protein aggregates, including amyloid-β(Aβ) 42 and its toxic oligomers; increased y-secretase activity; and impaired autophagy. Cultured human muscle fibers with experimentally-impaired autophagy recapitulate some of the s-IBM muscle abnormalities, including vacuolization and decreased activity of lysosomal enzymes, accompanied by increased Aβ42, Aβ42 oligomers, and increased γ-secretase activity. NaPB treatment reverses lysosomal dysfunction in an in vitro model of inclusion-body myositis, involving cultured human muscle fibers. NaPBA treatment improved lysosomal activity, decreased Aβ42 and its oligomers, decreased γ-secretase activity, and virtually prevented muscle-fiber vacuolization.

However, it is difficult to monitor a patient with s-IBM being treated with PBA. Thus, there is a need to provide for treating the subject with s-IBM and monitoring the patient receiving the PBA treatment.

### SUMMARY

In some embodiments, a method for treating inclusion body myositis (IBM) in a subject can include: providing a subject that has IBM; administering phenylbutyrate (PBA) to the subject in a therapeutically effective amount to provide a treatment for the IBM; obtaining at least one biological sample from the subject after initiation of the treatment for the IBM; measuring a biological indicator in the at least one biological sample, wherein the biological indicator is mitochondrial membrane potential; and monitoring a change in the biological indicator as an indication of effectiveness of the treatment.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A illustrates mitotracker deep red 633.

FIG. 1B illustrates mitotracker orange.

FIG. 1C illustrates mitotracker red fm.

FIG. 1D illustrates mitotracker green fm.

FIG. 1E illustrates mitotracker Red CMXROS.

FIG. 1F illustrates mitotracker green.

FIGS. 2A-2E include graphs that show data for the subjects, which indicates safety for the PBA administration.

FIGS. 3A-3B include data that shows the IBMFRS-Inclusion body myositis functional rating scale.

FIGS. 4A-4B include data that shows the TUG (seconds) over the duration of the treatment.

FIGS. 5A-5F includes data that shows a strength evaluation of 24 muscles with accumulative total score. No meaningful change is shown.

FIGS. 6A-6B includes data that shows knee extension and hand grip measurements, which provide another test of muscle strength of certain muscles using a device (dynamometer) instead of manual testing.

FIGS. 7A-7B include data that shows the amount of Annexin, which is an apoptosis (cell death) marker.

FIGS. 8A-8B include data that shows the amount of MitoSox, which is a mitochondrial biomarker which detects mitochondrial superoxide levels.

FIGS. 9A-9B show the amount of tetramethylrhodamine, ethyl ester (TMRE) is a cell-permeant, cationic, red-orange fluorescent dye that is readily sequestered by active mitochondria.

FIGS. 10A-10B show that the TAR DNA binding protein 43 (TDP-43) accumulates in muscles of patients with IBM and can be measured in the blood.

FIGS. 11A-11B show the MitoTracker (mitochondrial tracker) data, which provides information about the overall mitochondrial mass, which changes with treatment with PBA.

FIGS. 12A-12B show the statistical analysis, which showed statistically significant change in MitoTracker (mitochondrial mass).

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology includes phenylbutyrate (PBA) and pharmaceutical compositions having PBA for the use in a therapy for inclusion body myositis (IBM). The PBA can be used safely and efficiently in the treatment of IBM as shown in the data provided herein. PBA is an orally active chemical chaperone that has been previously approved by the U.S. Food and Drug Administration (FDA) for treatment of urea cycle disorders. The PBA may mimic the function of intracellular molecular chaperones in preventing protein aggregation and oligomerization, which is thought to be the main disease mechanism of IBM and other neurodegenerative disorders. As such, the PBA may prevent the protein aggregation and oligomerization in the treatment of IBM. There are currently no effective or approved therapies available for patients with IBM. Now, PBA can be used in treating IBM as described herein.

In a study, PBA at a dose of 3 grams (gm) was administered twice daily in patients with inclusion body myositis. In this study, 10 patients received the daily doses of PBA for 3 months after run-up periods of 3 months. The medication was well tolerated, and no serious adverse events related to the study drug were reported. The study was conducted by monitoring biomarkers and biological indicators in the subjects. One of the biological indicators is mitochondrial membrane potential, and analyzing the biological indicator uses a mitochondrial tracker (e.g., MitoTracker) to monitor mitochondrial membrane potential. The use of the mitochondrial tracker showed statistically significant change in the mitochondria from PBA suggesting target engagement and use of PBA as a therapy for treatment of IBM. As such, the biomarkers, including a mitochondrial tracker, allow for monitoring the progress of treatment of IBM in the subject with PBA. Thus, the present invention can use PBA in human subjects to treat inclusion body myositis with a dose of 3 gm twice daily and monitoring biological indicators or other biomarkers to monitor progress of IBM in the subject.

A class of proteins called molecular chaperones has evolved to prevent inappropriate interactions within and between non-native polypeptides, to enhance the efficiency of de novo protein folding, and to promote the refolding repair of proteins that have become misfolded as a result of cellular stress. In addition to this protein repair activity, chaperones can mediate targeting to the proteasome system or to lysosomes, resulting in selective degradation of the misfolded protein when the chaperones cannot repair the misfolded proteins.

The PBA is a histone deacetylase inhibitor, which is converted/oxidized in vivo into phenylacetate (PAA) by beta-oxidation. PBA can also mimic the function of intracellular molecular chaperones in preventing protein aggregation and oligomerization. Phenylbutyrate has activity in lysosomal activity-inhibited CHMFs where it substantially: (a) improves the phenotype of muscle-fibers by decreasing their vacuolization; (b) increased cathepsins D and B activities, accompanied by a decrease of NBR1, p62 and LC3-II; (c) decreases Aβ42 and Aβ42 oligomers; and (d) decreases γ-secretase activity. This improvement of lysosomal activity and striking ameliorative consequences of experimentally-impaired autophagy provide for phenylbutyrate as a drug for IBM (Nogalska A, et al.; Sodium phenylbutyrate reverses lysosomal dysfunction and decreases amyloid-β42 in an in vitro model of inclusion-body myositis, Neurobiol. Dis 2014;65:93-101).

In some embodiments, subjects having IBM can be treated with a dosing regimen of PBA. The dosing regimen can include from about 1 gram (gm) to 21 gm of PBA a day, which can be in a single or multiple doses. The daily dose can range up to 20 or 21 grams a day, or up to 15 grams a day, or up to 10 grams a day, or up to 9 grams a day, or up to 8 grams a day, or up to 6 grams a day. The dosing regimen can be in a single dose, two doses, three doses, or more in order to arrive at the daily dose. An example includes two 3 gm doses twice per day.

The mitochondrial tracker used to track treatment of IBM with PBA can include MitoTracker dyes that are cationic fluorophores that accumulate electrophoretically into mitochondria in response to the highly negative mitochondrial membrane potential. MitoTracker dyes possess a reactive chloromethyl group that forms a covalent bond with thiols on proteins and peptides, which traps MitoTracker dyes within mitochondria. Thus, mitochondria retain MitoTracker dyes like MitoTracker Orange CMTMRos after loss of their membrane potential. Sec, Kholmukhamedov A, Schwartz J M, Lemasters J J. Isolated mitochondria infusion mitigates ischemia-reperfusion injury of the liver in rats: mitotracker probes and mitochondrial membrane potential. Shock. 2013 June; 39(6):543. doi:10.1097/SHK.0b013e318292300d. PMID: 23680774; PMCID: PMC3759289.

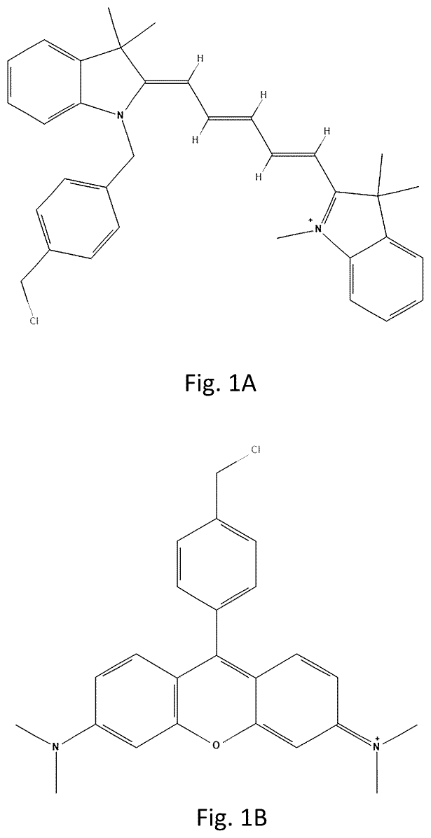

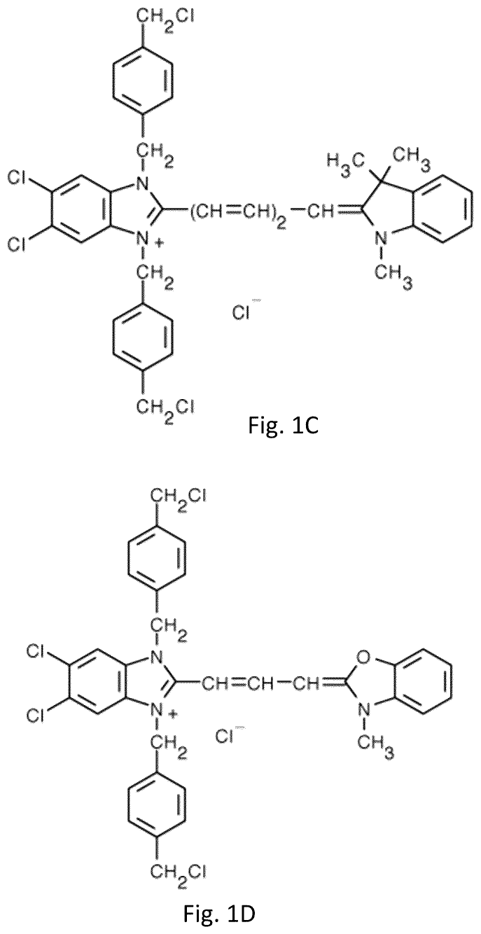

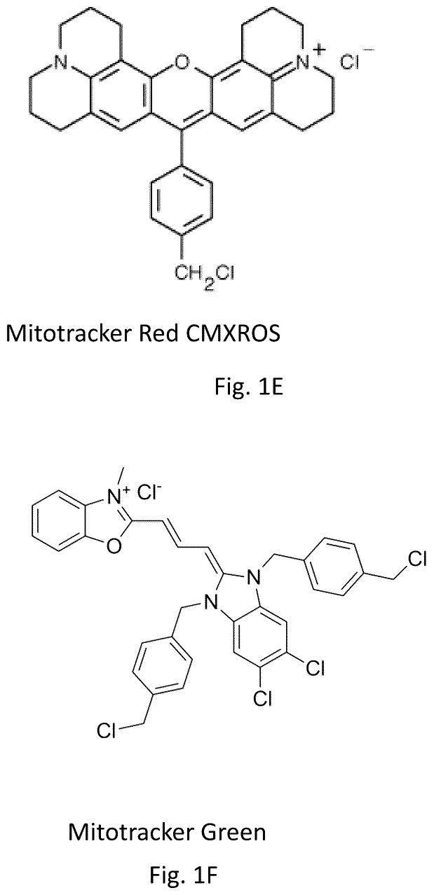

An example is mitotracker deep red 633 as show in FIG. 1A, also known as (2Z)-1-[[4-(chloromethyl)phenyl]methyl]-3,3-dimethyl-2-[(2E,4E)-5-(1,3,3-trimethylindol-1-ium-2-yl)penta-2,4-dienylidene]indole;chloride.

Another example is mitotracker orange as shown in FIG. 1B, also known as [9-[4-(chloromethyl)phenyl]-6-(dimethylamino)xanthen-3-ylidene]-dimethylazanium;chloride.

Another example is mitotracker red fm as shown in FIG. 1C.

Another example is mitotracker green fm as shown in FIG. 1D.

Another example is mitotracker Red CMXROS as shown in FIG. 1E.

Another example is mitotracker green as shown in FIG. 1F.

In some embodiments, the term “mitochondrial tracker” as used herein includes a mitotracker. The mitochondrial tracker can be overall cationic and include a reporter segment (e.g., fluorophore) and a reactive chloromethyl group that forms a covalent bond with thiols on proteins and peptides inside of the mitochondria.

Pharmaceutically acceptable salts of compounds described herein, such as PBA, are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

### EXAMPLES

Safety and tolerability of PBA in IBM patients were studied, with a run-in period of 3 months without medication. Biomarkers were studied. These biomarkers evaluate the mitochondrial function, stress response, degenerative process and apoptosis and include mitochondrial membrane potentials through MitoTracker red (shows overall mitochondrial mass), MitoSox (a mitochondrial biomarker which detects mitochondrial superoxide levels), tetramethylrhodamine ethyl ester (determine mitochondrial membrane potential and a marker of overall mitochondrial activity), Annexin binding in lymphocytes (apoptosis marker). We have previously assayed TDP-43 and its derivative, phosphorylated TDP-43 (pTDP-43) in peripheral blood platelets in our laboratory (Dr. Agbas). TDP43 positive cytoplasmic aggregates have been described in ALS motor neurons and IBM skeletal muscle tissue with significant up-regulation of TARDBP (the gene expresses TDP-43 protein) and SQTM1 (Sequestosome-1) gene expression.14 Therefore, we planned to measure blood-derived platelet total TDP-43 and pTDP-43 as surrogate biomarker for skeletal muscle TDP-43 profile of IBM patients. Our laboratory had demonstrated analytical capability of the measurement of platelet TDP-43 and pTDP-43 in human platelets.^{13,14,15}

Primary outcome measures included adverse event reporting. Secondary outcome measures included multiple strength and functional measures: manual muscle testing, timed up and go test (TUG), IBM functional rating scale (IBMFRS), and grip strength. These clinical outcome measures were commonly used in evaluating progression of IBM and in clinical trials of this disease.^{18,19,20,21} In addition, safety laboratory tests and electrocardiogram would be monitored regularly. As mentioned above, the biomarkers would be repeated at the end of the treatment period.

Table 1 shows the patient status for the studies.

| | Number |
|---|---|
| Patient Status | |
| Enrolled | 11 |
| Sex | |
| Female | 7 |
| Male | 4 |
| Ethnicity | |
| Hispanic or Latino | 1 |
| Non-Hispanic/Latino | 10 |
| Race | |
| Caucasian | 10 |
| Other | 1 |

The study drug was well tolerated, and all enrolled subjects were able to continue it throughout the treatment period. There was one serious event (a fall that required a hospital visit) that was not related to the study drug. No study drug-related serious events were reported. Two subjects had mildly elevated liver enzymes at baseline before the treatment period. No other significant laboratory changes were noted. One subject had hair loss which was considered possibly related. The most common adverse events were related to gastrointestinal symptoms (n=9), (Tables 2 and 3).

TABLE 2
Adverse Events (considered possibly or probably related)

| Adverse Events (possibly | | Number of | Number of |
|---|---|---|---|
| or probably | | occurrences | subjects |
| Gastrointestinal AEs | | 9 | 9 |
| | Heartburn | 4 | 4 |
| | Nausea | 2 | 2 |
| | Urgency | 1 | 1 |
| | Constipation | 1 | 1 |
| | Oral Aphthous | 1 | 1 |
| Musculoskeletal Pain | | 2 | 2 |
| Drowsiness | | 1 | 1 |
| Tremor | | 1 | 1 |
| Hair Loss | | 1 | 1 |
| Two Itchy Skin Lesions | | 1 | 1 |
| Nasal Congestion | | 1 | 1 |

TABLE 3
Adverse Events (considered unrelated)

| | Number of | Number of |
|---|---|---|
| Adverse Events (considered | occurrences | subjects |
| Falls | 9 | 6 |
| Urinary Urgency | 2 | 2 |
| Elevated blood pressure | 2 | 2 |
| Elevated liver enzymes | 2 | 2 |
| Low blood pressure | 1 | 1 |
| Headache | 1 | 1 |
| Chills | 1 | 1 |
| Numbness in the Legs | 1 | 1 |
| Back Pain | 1 | 1 |
| Elevated White Blood Count | 1 | 1 |
| Right Bundle Branch Block | 1 | 1 |
| Strep. Throat Infection | 1 | 1 |

The PBA was administered orally at 3 gm doses twice a day. The data shows that the PBA was well tolerated, and all enrolled subjects were able to continue it throughout the treatment period. No study drug related serious events were reported. Mildly elevated liver enzyme reported in two subjects at baseline and before the treatment period. The most common adverse events which were considered related were GI symptoms (n=9).

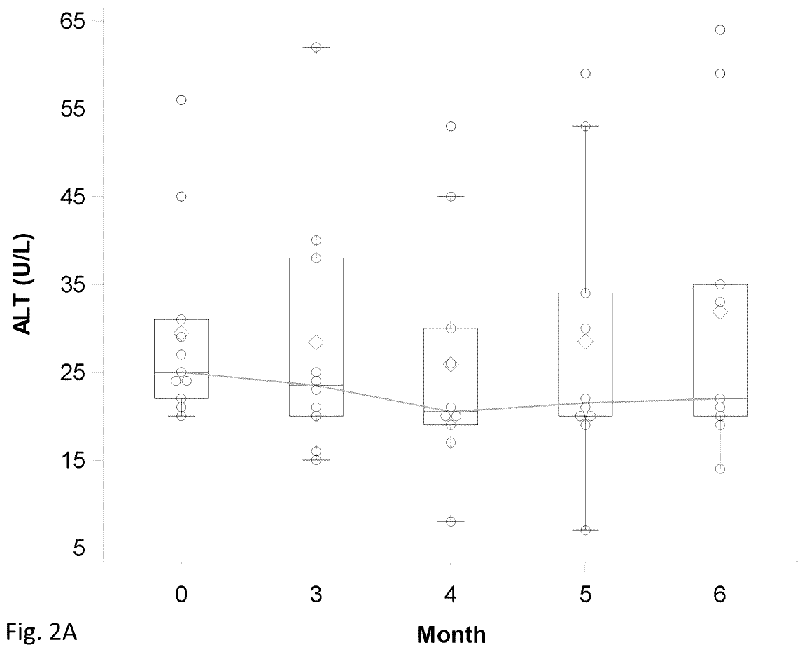

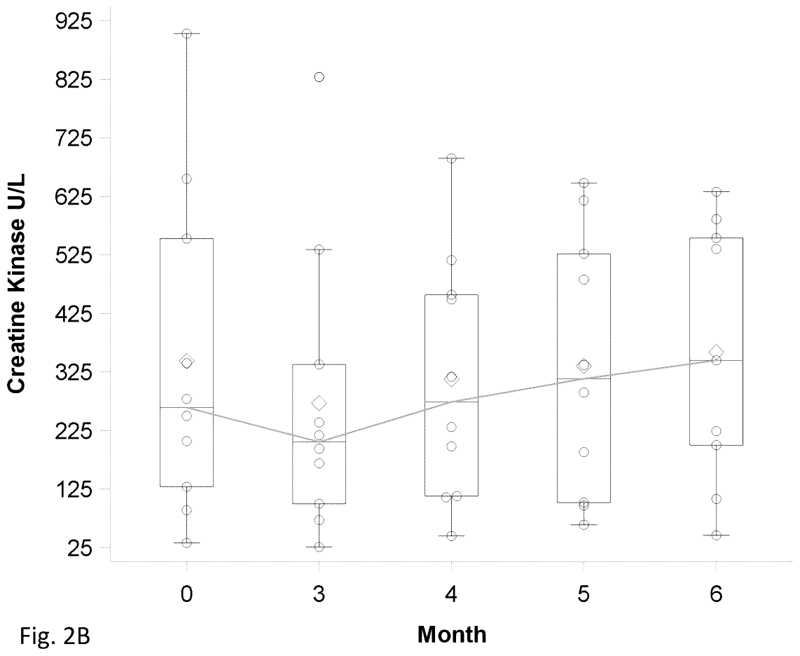

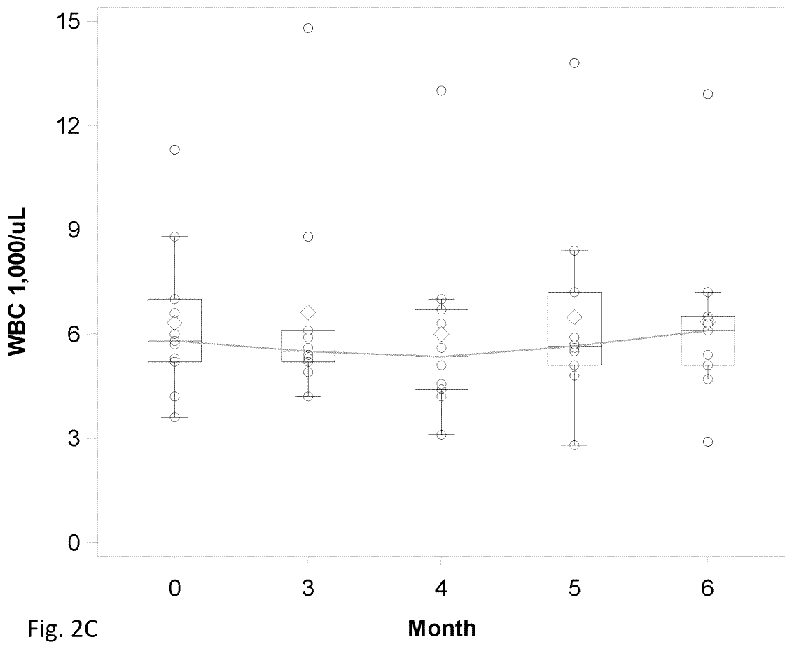

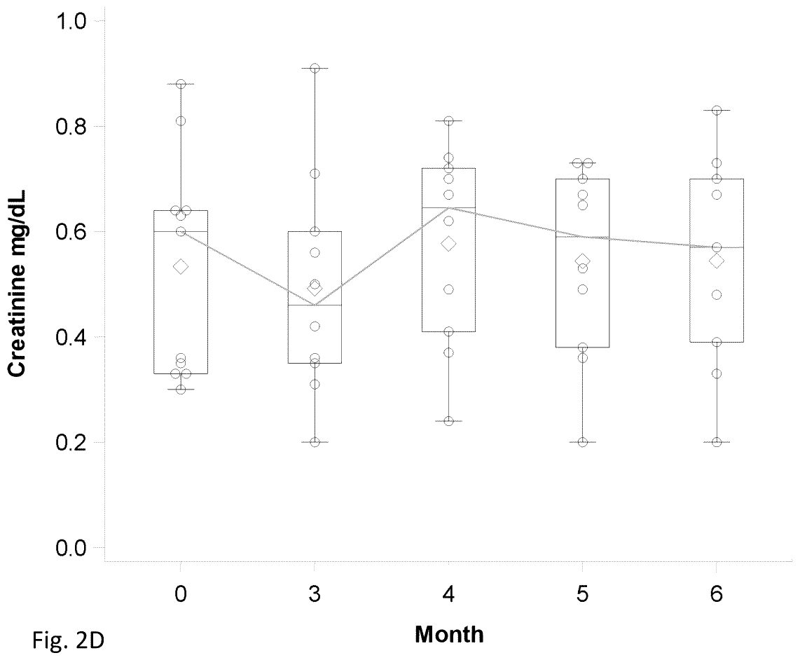

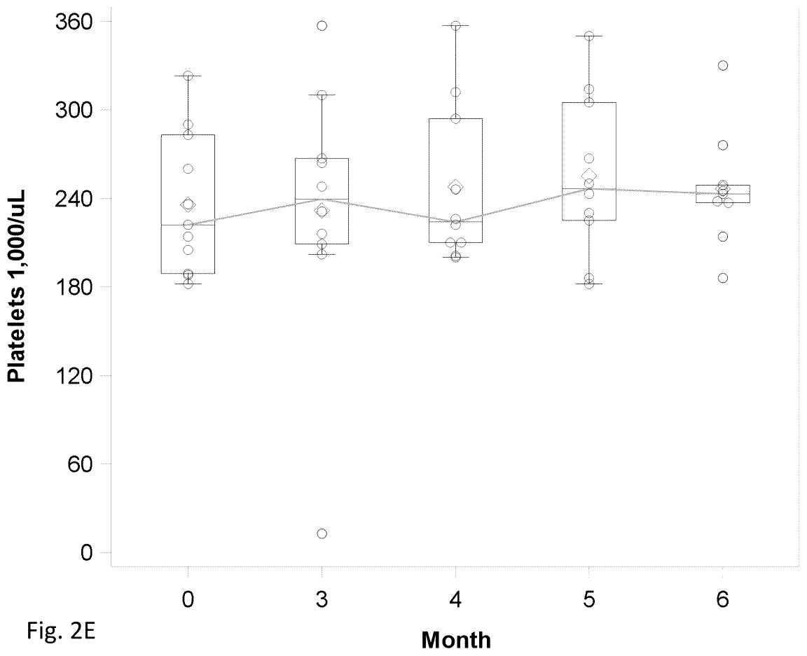

FIGS. 2A-2E show data for the subjects, which indicates safety for the PBA administration. The safety lab tests for monitoring purposes include: Liver enzyme (ALT), renal function test (creatinine), muscle enzyme (creatine kinase) and blood counts (including WBC and platelets).

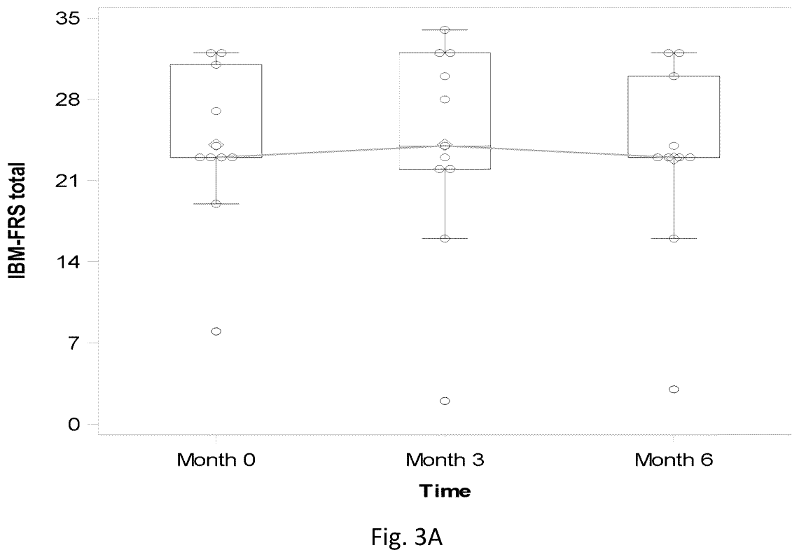

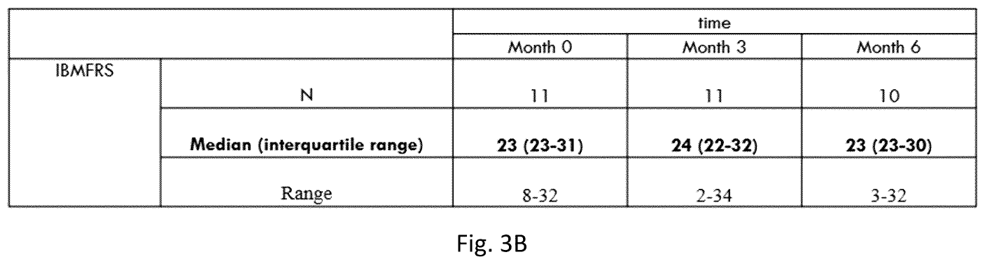

FIGS. 3A-3B show the IBMFRS—Inclusion body myositis functional rating scale. This scale helps monitor the disease progression over time. The IBMFRS did not seem to change during the study (Median was 23 at baseline, 24 at the beginning of the treatment period, and 23 at the end of it), (FIG. 3A).

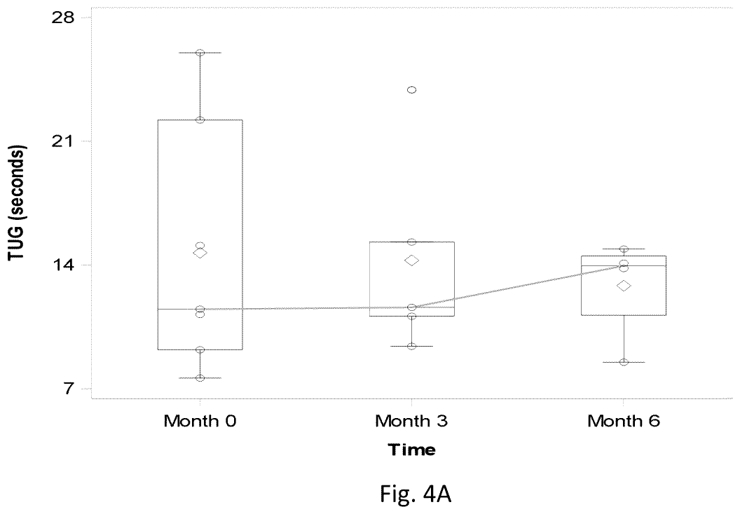

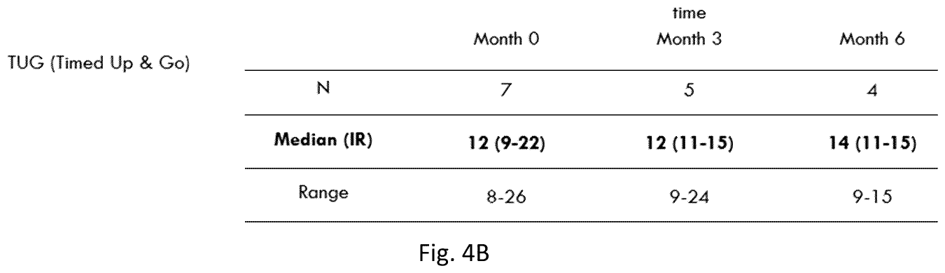

FIGS. 4A-4B show the TUG (seconds) over the duration of the treatment. No meaningful change is shown.

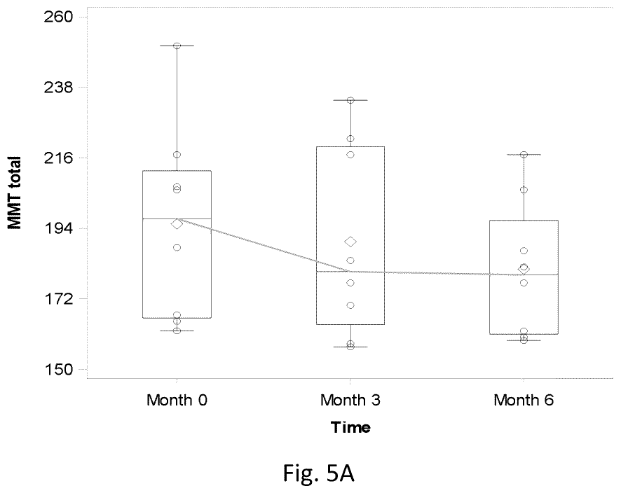

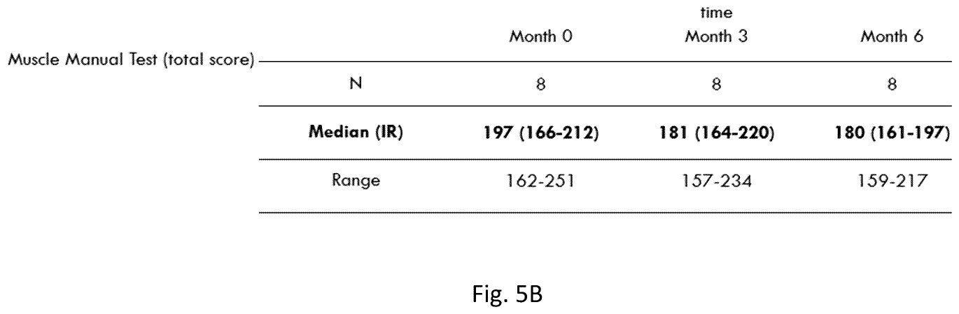

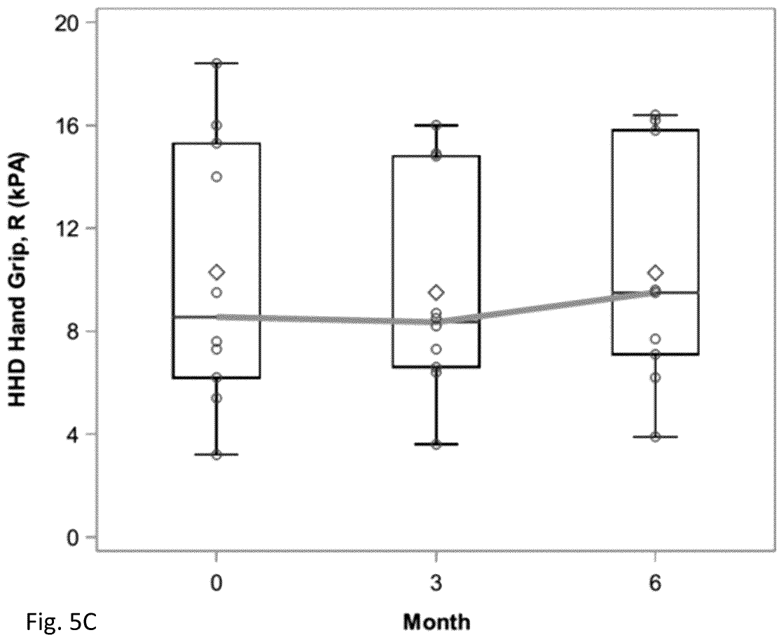

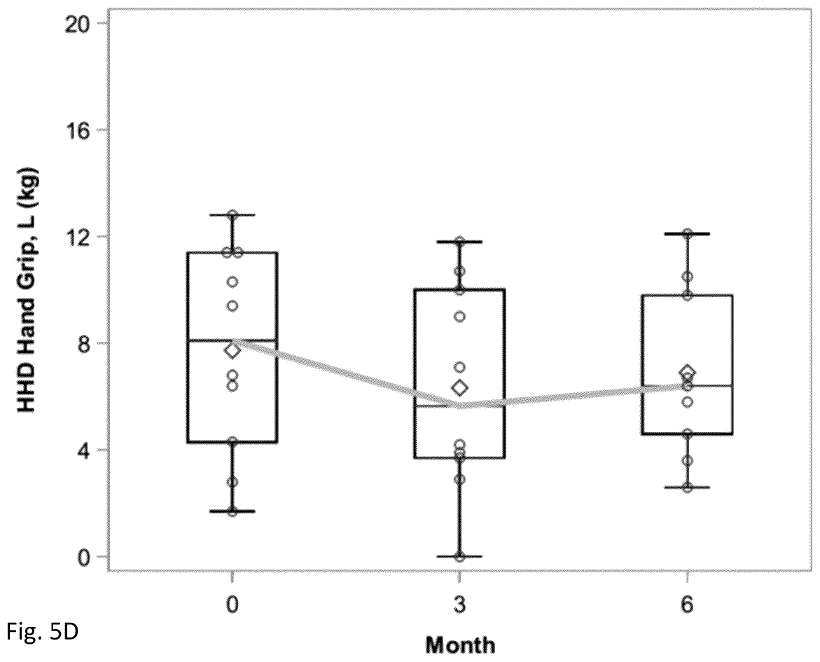

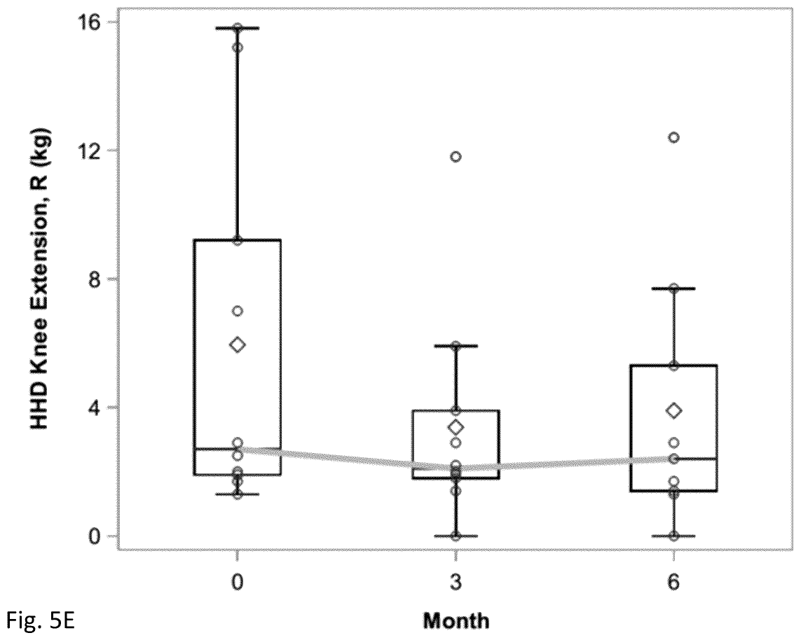

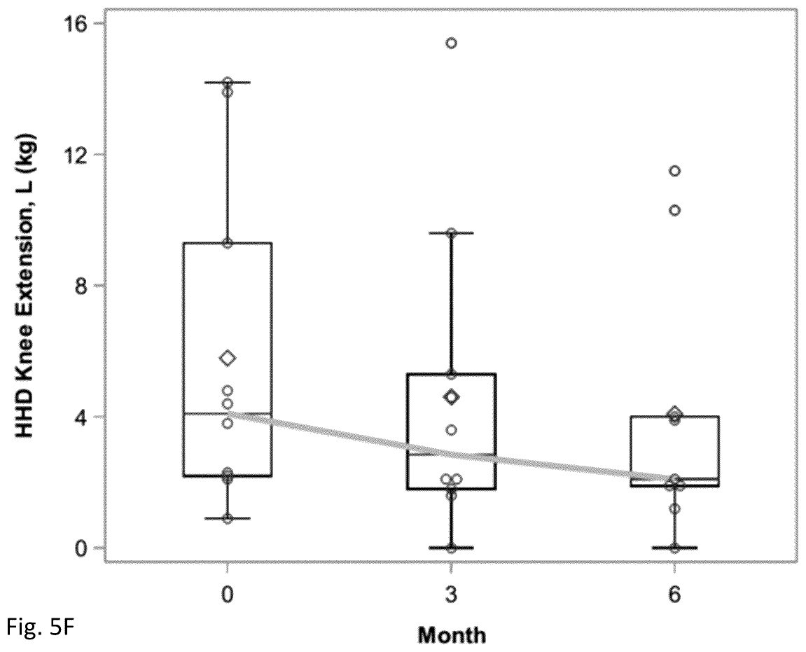

FIGS. 5A-5F show a strength evaluation of 24 muscles with accumulative total score. No meaningful change is shown.

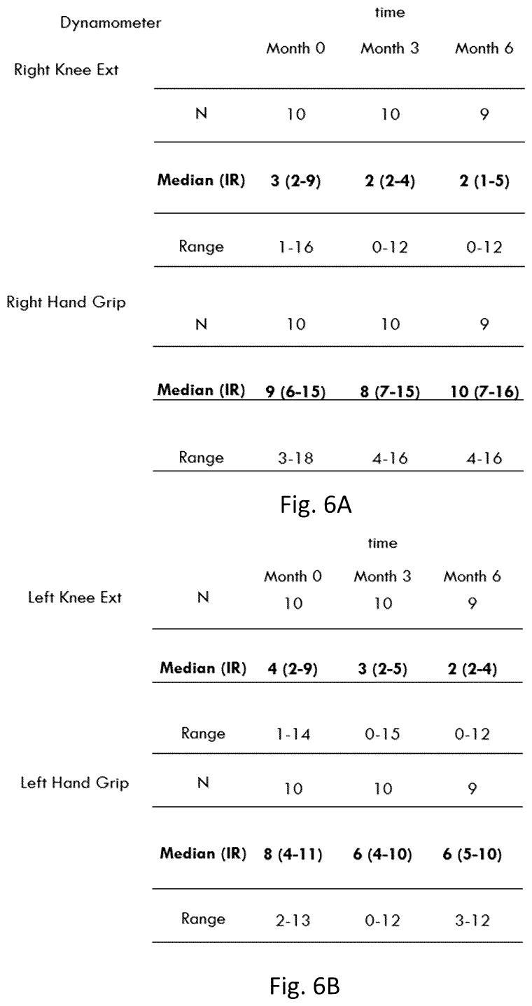

FIGS. 6A-6B show knee extension and hand grip measurements, which provide Another test of muscle strength of certain muscles using a device (dynamometer) instead of manual testing.

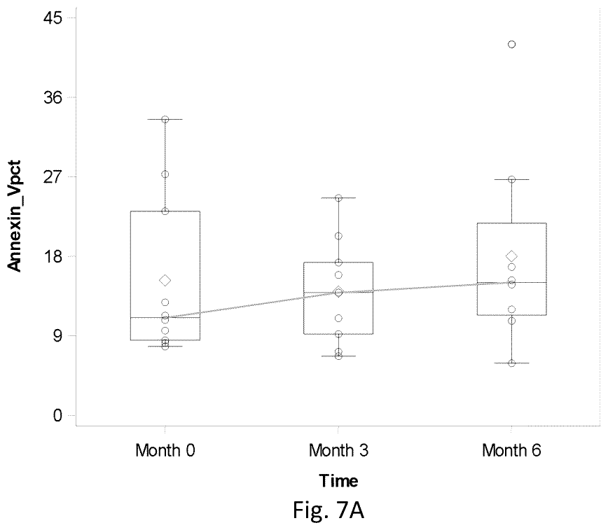

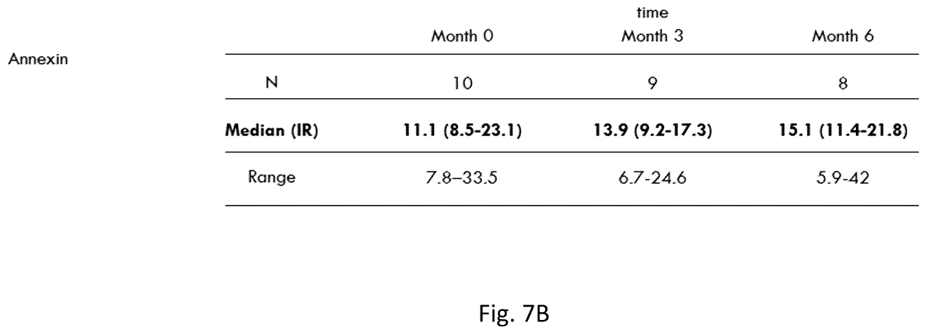

FIGS. 7A-7B show the amount of Annexin, which is an apoptosis (cell death) marker.

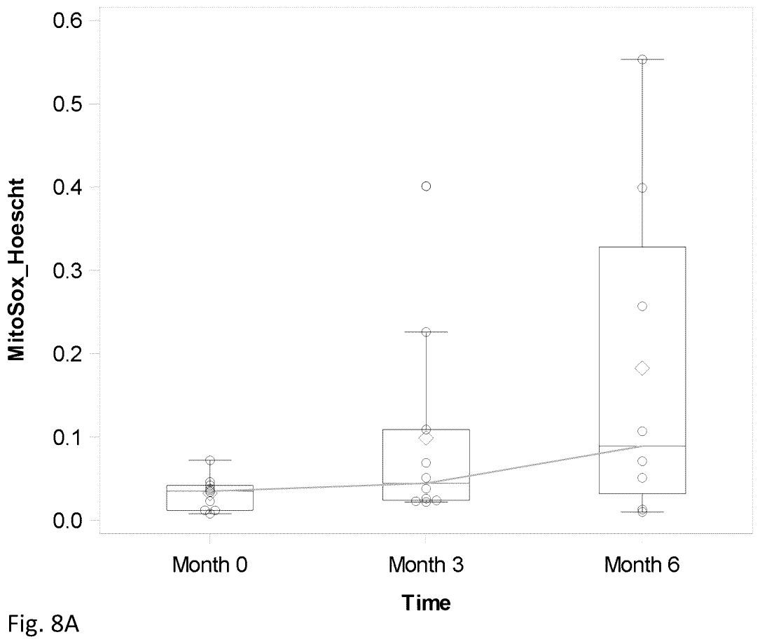

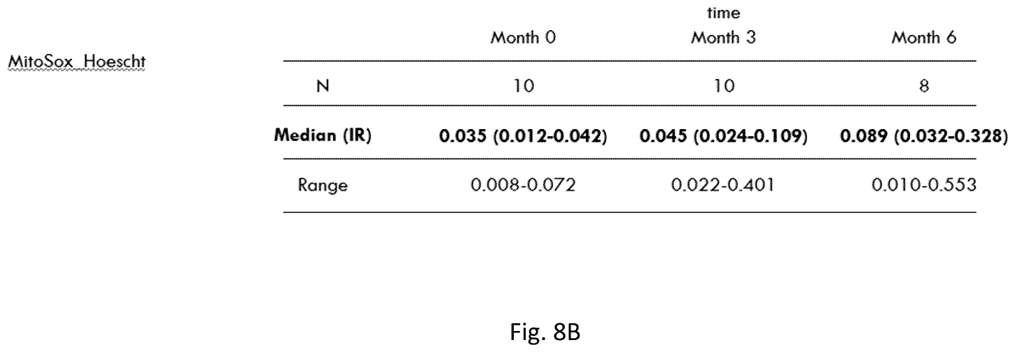

FIGS. 8A-8B show the amount of MitoSox, which is a mitochondrial biomarker which detects mitochondrial superoxide levels. Superoxide is a form of reactive oxygen species.

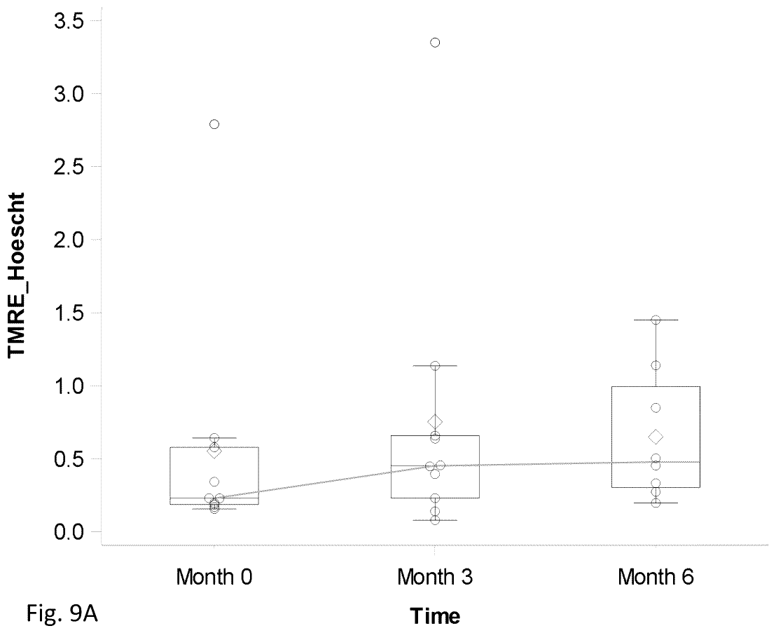

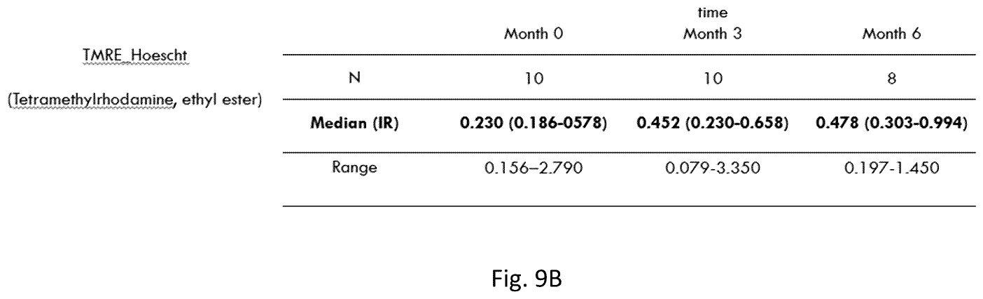

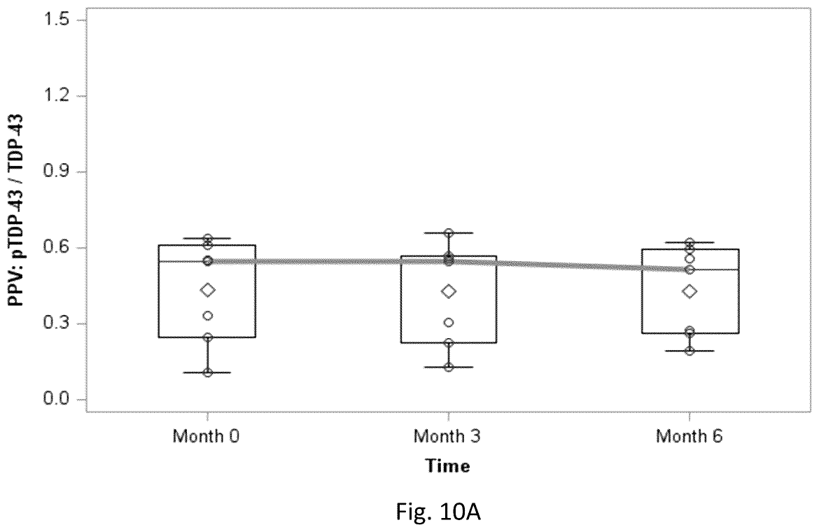

FIGS. 9A-9B show the amount of tetramethylrhodamine, ethyl ester (TMRE) is a cell-permeant, cationic, red-orange fluorescent dye that is readily sequestered by active mitochondria. The TMRE determines the mitochondrial membrane potential and is a marker of overall mitochondrial activity.

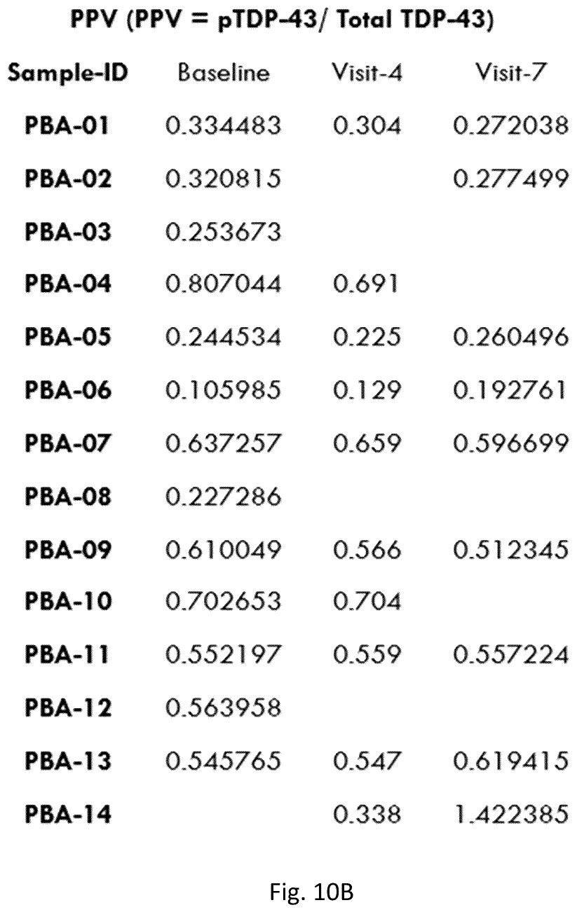

FIGS. 10A-10B show that the TAR DNA binding protein 43 (TDP-43) accumulates in muscles of patients with IBM and can be measured in the blood.

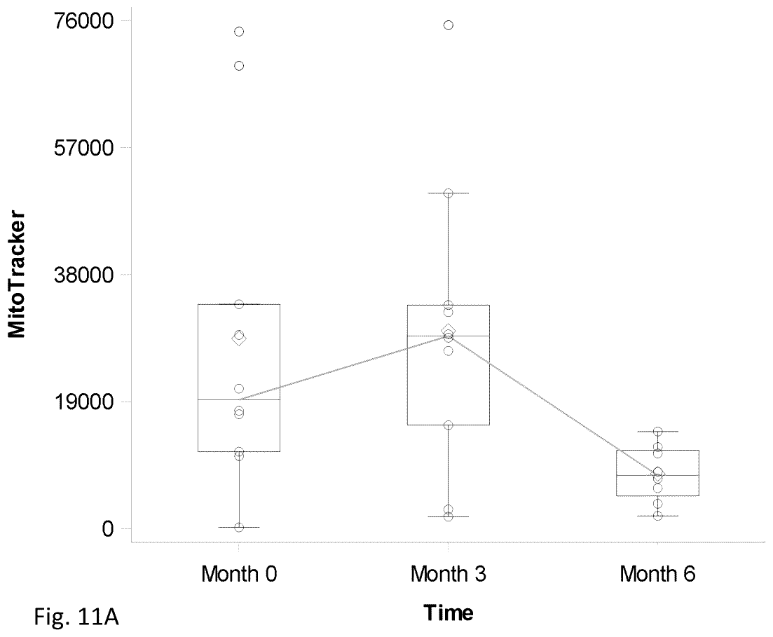

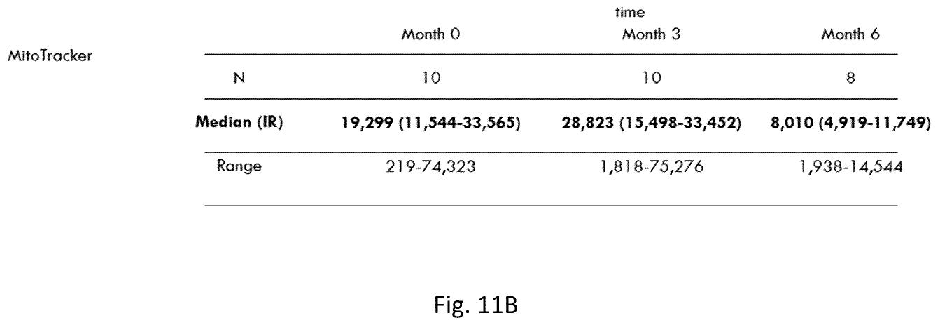

FIGS. 11A-11B show the MitoTracker (mitochondrial tracker) data, which provides information about the overall mitochondrial mass, which changes with treatment with PBA. This indicates the mitochondrial trackers can be used for tracking improvement of IBM with treatment of PBA.

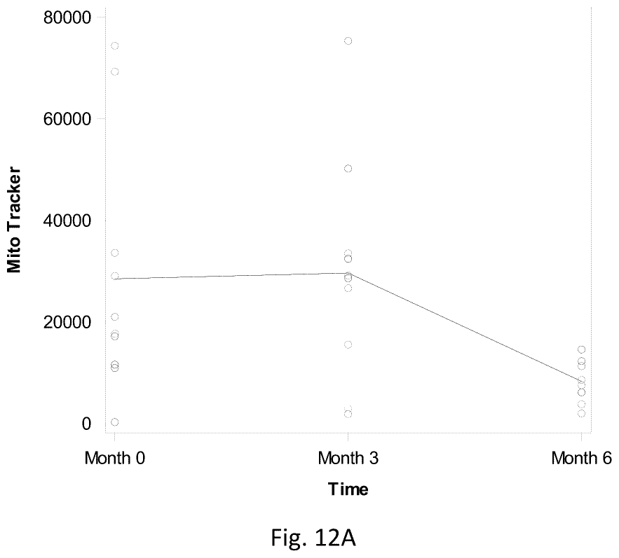

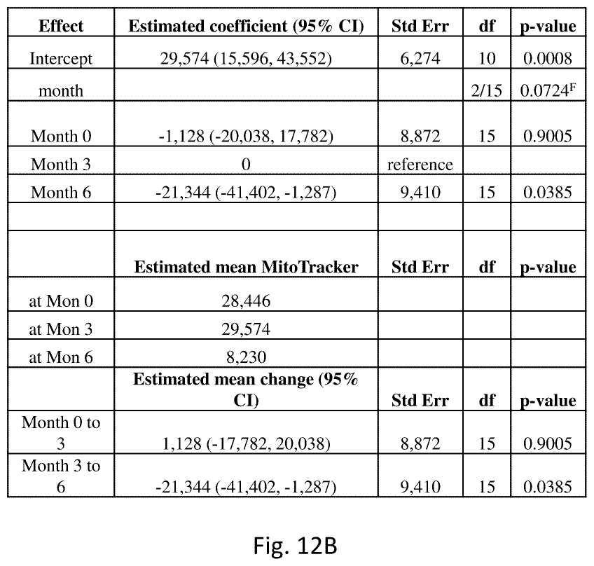

FIGS. 12A-12B show the statistical analysis, which showed statistically significant change in MitoTracker (mitochondrial mass). Thus, the mitochondrial tracker can be used to track treatment of IBM.

While there was no meaningful change in most exploratory biomarkers used in this study (FIGS. 7A-7B, 8A-8B, 9A-9B, 10A-10B), the MitoTracker dropped at the end of the treatment period (median of 8010 comparing to 19299 at the baseline and 28823 at the beginning of the treatment period (FIGS. 11A-11B). This was investigated further with a statistical analysis (detailed below in Table 4 and FIG. 12A) which showed that the mean change between the run-in period and treatment period was statistically significant (p=0.0243).

| | Estimated coefficient | Std | | |
|---|---|---|---|---|
| Effect | (95% CI) | Err | df | p-value |
| Intercept | 29,574 (15,596, 43,552) | 6,274 | 10 | 0.0008 |
| Month | | | 2/15 | 0.0724^{F} |
| Month 0 | −1,128 (−20,038, 17,782) | 8,872 | 15 | 0.9005 |
| Month 3 | 0 | reference | | |
| Month 6 | −21,344 (−41,402, −1,287) | 9,410 | 15 | 0.0385 |
| On treatment - | 20,780 (3,091, 38,469) | 8299 | 15 | 0.0243^{t} |
| Off treatment | | | | |

### Results

Box plots of outcomes provide for visual inspection of the observed values as well as a representation of the median, inter-quartile range, and mean.

The trend observed in the boxplots of MitoTracker data warranted further exploration with a post hoc analysis. A generalized linear mixed model was used to estimate mean values of MitoTracker at each timepoint. 10 of 11 subjects had measurements at all 3 timepoints. Time was used as a categorical value and correlation among an individual's repeated measurements was accounted for with a random effect for subject. The fixed effect, month, was not statistically significant (p=0.0724). Although, MitoTracker values after treatment are statistically significantly different from pre-treatment, months 0 and 3 combined (p=0.0243). The results from the model are exploratory.

This is the first clinical trial of phenylbutyrate (PBA) in inclusion body myositis (IBM), and it was justified by the potential benefits of PBA in IBM as detailed above. It was found that PBA is safe and well-tolerated in subjects with IBM. There were no concerning safety findings in this study and the only serious event reported (a fall) was related to the disease itself, not the study drug. There were no meaningful changes in all clinical and most biological outcome measures. This was not unexpected considering the short duration of this study and the small sample size. Despite these limitations, the MitoTracker showed a significant drop with treatment. Reduced MitoTracker with treatment could be due to reduced mitochondrial number/mass. The data indicates that PBA induces autophagy/mitophagy as it is known to reduce protein aggregation and one way to process aggregates is to ship them to mitochondria and then induce mitophagy.

In some embodiments, studies can be performed to verify the MitoTracker change and to further evaluate for any clinical or biological outcome measures changes that might have been missed due to the small sample size and short duration of our pilot study. Studying the muscle tissue should be considered in any future trial to evaluate for histopathological changes secondary to treatment with PBA considering the changes found in the muscle cell model of IBM when exposed to PBA.¹³

Phenylbutyrate was safe and well tolerated in patients with IBM in this small pilot study. The change in the MitoTracker suggests target engagement, Thus, MitoTracker can be used to track the therapy for effectiveness of the treatment.

### Study Eligibility

Inclusion Criteria: Fulfill ENMC 2011 diagnostic criteria for IBM; Age≥18 years; Women must be post-menopausal (no menses in >12 months) or status post hysterectomy; Able to give informed consent; Subjects must be able and willing to remain on stable concomitant medications throughout the duration of the study.

Exclusion Criteria: Presence of any one of the following medical conditions: chronic infection; chronic renal insufficiency; cancer other than skin cancer less than five years prior; multiple sclerosis or prior episode of central nervous system demyelination; or other chronic serious medical illnesses; Presence of any of the following on routine blood screening: WBC<3000; Platelets<100,000; hematocrit<30%; BUN>30 mg/dl; creatinine>1.5 mg/dl; liver disease with serum albumin<3 g/dl; Women who are pregnant or lactating; History of non-compliance with other therapies; Coexistence of other muscular disease; Drug or alcohol abuse within the past three months; Known bleeding disorder; Known liver disease; Known congestive heart failure; Known hypernatremia.

### Statistics

For the primary outcome of safety, the frequency of Adverse Events (AEs) and Serious Adverse Events (SAEs) by grade, would be reported. This would be done for each follow-up visit as well as for all visits combined.

For the secondary outcomes, the measurements at baseline, month-3, and month-7 would be reported using mean and standard deviation (or median and interquartile range for skewed measurements) along with a corresponding 95% confidence interval. These measurements would be visually compared using box plots to detect the presence of a trend over time by drawing a trend line on the means (or median). Due to the small sample size, this comparison would be done visually, and no formal statistical test would be done routinely but it would be considered to further study a trend.

### Embodiments

Embodiment 1. A method for treating inclusion body myositis (IBM) in a subject, comprising: providing a subject that has IBM; administering phenylbutyrate (PBA) to the subject in a therapeutically effective amount to provide a treatment for the IBM; obtaining at least one biological sample from the subject after initiation of the treatment for the IBM; measuring a biological indicator in the at least one biological sample, wherein the biological indicator is mitochondrial membrane potential; and monitoring a change in the biological indicator as an indication of effectiveness of the treatment.

Embodiment 2. The method of one of the embodiments, comprising: obtaining at least one blood sample as the at least one biological sample; measuring mitochondrial membrane potential of lymphocytes in the at least one blood sample; and determining whether the mitochondrial membrane potential of the lymphocytes has changed in response to the PBA.

Embodiment 3. The method of one of the embodiments, comprising: at least partially purifying the lymphocytes from the biological sample; incubating a marker for mitochondrial membrane potential assessment with the lymphocytes in a medium; removing excess marker from the medium with the lymphocytes; and measuring the marker retained in the mitochondria of the lymphocytes.

Embodiment 4. The method of one of the embodiments, wherein the marker is a cationic fluorophore.

Embodiment, 5. The method of one of the embodiments, wherein the marker accumulates electrophoretically into mitochondria via negative membrane potential.

Embodiment 6. The method of one of the embodiments, wherein the marker includes a reactive chloromethyl group that forms a covalent bond with thiols on proteins and peptides inside of the mitochondria.

Embodiment 7. The method of one of the embodiments, wherein the marker includes a mitochondrial tracker that can be overall cationic with a fluorescent reporter segment and a reactive chloromethyl group.

Embodiment 8. The method of one of the embodiments, comprising: obtaining at least one initial biological sample from the subject before initiation of the treatment for the IBM; measuring the biological indicator in the at least one initial biological sample; and monitoring a change in the biological indicator from the at least one initial biological sample to the at least one biological sample as an indication of effectiveness of the treatment.

Embodiment 9. The method of one of the embodiments, wherein the administration includes from about 1 gram (gm) to 10 gm of PBA a day.

Embodiment 10. The method of one of the embodiments, wherein the administration is daily for at least one week.

Embodiment 11. The method of one of the embodiments, wherein the administration is for at least one month.

Embodiment 12. The method of one of the embodiments, wherein the administration is for at least three months.

Embodiment 13. The method of one of the embodiments, comprising: establishing an initial mitochondrial membrane potential prior to the treatment; and measuring the mitochondrial membrane potential from a plurality of biological samples taken over a period of time, such as at least one week, at least one month, or at least three months.

Embodiment 14. The method of one of the embodiments, comprising: measuring Bcl2 in an RNA sample of the at least one biological sample; measuring Bax in the RNA sample of the at least one biological sample; and determining a Bcl2/Bax expression ratio in the RNA sample prepared from lymphocytes of each of the at least one biological sample.

Embodiment 15. The method of one of the embodiments, comprising: measuring an apoptosis marker in an initial biological sample obtained prior to the treatment; measuring the apoptosis marker in the at least one biological sample; and monitoring changes in the amount of apoptosis marker after initiation of the treatment.

Embodiment 16. The method of one of the embodiments, wherein the apoptosis marker is Annexin binding in lymphocytes of the at least one biological sample.

Embodiment 17. The method of one of the embodiments, comprising: measuring oxygen radical antioxidant capacity of an initial biological sample obtained prior to the treatment; measuring the oxygen radical antioxidant capacity in the at least one biological sample; and monitoring changes in the oxygen radical antioxidant capacity after the treatment.

Embodiment 18. The method of one of the embodiments, comprising determining the subject to have IBM.

Embodiment 19. The method of one of the embodiments, comprising screening the subject prior to the treatment, wherein the screening includes one or more of the following inclusion criteria: the subject has diagnostic criteria for IBM; the subject has an age ≥18 years, or optionally greater than 40 years or greater than 50 years; a woman subject must be post-menopausal, no menses in >12 months, or status post hysterectomy; or the subject commits to remain on stable concomitant medications throughout the duration of the treatment.

Embodiment 20. The method of one of the embodiments, comprising screening the subject prior to the treatment, wherein the screening includes one or more of the following exclusion criteria: presence of any one of the following medical conditions in the subject: chronic infection; chronic renal insufficiency; cancer other than skin cancer less than five years prior; multiple sclerosis or prior episode of central nervous system demyelination; or other chronic serious medical illnesses; presence of any of the following on routine blood screening of the subject: WBC<3000; Platelets<100,000; hematocrit<30%; BUN>30 mg %; creatine>1.5 mg %; or liver disease with serum albumin<3 G/DL; women who are pregnant or lactating; coexistence of other muscular disease in the subject; drug or alcohol abuse within past three months by the subject; bleeding disorder in the subject; liver disease in the subject; congestive heart failure in the subject; or hypernatremia in the subject.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only and is not intended to be limiting.

As used herein, the term “treat” or “treated” or “treating” or “treatment” refer to any type of action that imparts a modulating effect, which, for example, can be a beneficial effect, to a subject afflicted with a disorder, disease, or condition (e.g., IBM or symptoms), including improvement in the condition of the subject (e.g., in one or more symptoms), delay or reduction in the progression of the condition, and/or change in clinical parameters, disease or illness, etc., as would be well known in the art. The term “treatment” includes therapeutic and/or prophylactic treatment of IBM, the diminishment or alleviation of at least one symptom associated with IBM, and the eradication of one or more symptoms of IBM.

In view of the foregoing, the present technology provides pharmaceutical compositions and medicaments comprising any one or more of the compounds of the adjuvants, or derivative thereof, prodrug thereof, as disclosed herein and optionally a pharmaceutically acceptable carrier or one or more excipients or fillers. The compositions may be used in the methods and treatments described herein. Such compositions and medicaments include a therapeutically effective amount of any compound as described herein to function as a therapy for IBM. The pharmaceutical composition may be packaged in unit dosage form.

The pharmaceutical compositions and medicaments may be prepared by mixing one or more compounds of the present technology with pharmaceutically acceptable carriers, excipients, binders, diluents or the like. The compounds and compositions described herein may be used to prepare formulations and medicaments to treat IBM. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the present technology.

Those skilled in the art are readily able to determine an effective amount, such as by simply administering a compound of the present technology to a patient in increasing amounts of the adjuvant until the identification of a vaccine composition with the proper amount of adjuvant.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

### References

1. Felice K J, North W A. Inclusion body myositis in Connecticut: observation in 35 patients during an 8-year period. Medicine (Baltimore) 2001:80:320-7
2. Wilson F C, Ytterberg S R, St Sauver J L, Reed A M. Epidemiology of sporadic inclusion body myositis and polymyositis and Olmsted County, Minnesota. J Rheumatol. 2008:35:445-7
3. Dimachkie M M, Barohn R J. Inclusion body myositis. Curr Neurol Neurosci Rep. 2013;13:321
4. Machado P M, Dimachkie M M, Barohn R J. Sporadic inclusion body myositis: new insights and potential therapy. Curr Opin Neurol. 2014 October; 27(5):591-8.
5. Askanas V, Engel W K. Inclusion-body myositis: a myodegenerative conformational disorder associated with Abeta, protein misfolding, and proteasome inhibition. Neurology 2006; 66(2 Suppl 1):S39-48
6. Nogalska A, D'Agostino C, Engel W K, Klein W L, Askanas V. Novel demonstration of amyloid-β oligomers in sporadic inclusion-body myositis muscle fibers. Acta Neuropathol 2010; 120(5):661-6
7. Nogalska, A., D'Agostino, C., Terracciano, C., Engel, W. K., Askanas, V., 2010b. Impaired autophagy in sporadic inclusion-body myositis and in endoplasmic reticulum stress-provoked cultured human muscle fibers. Am. J. Pathol 2010; 177:1377-1387
8. Nogalska A, D'Agostino C, Engel W K, Askanas V. Activation of the γ-secretase complex and presence of γ-secretase-activating protein may contribute to Aβ42 production in sporadic inclusion-body myositis muscle fibers. Neurobiol Dis. 2012; 48(1):141-9
9. Forloni G, Terreni L, Bertani I et al. Protein misfolding in Alzheimer's and Parkinson's disease: genetics and molecular mechanisms. Neurobiol Aging. 2002; 23:957-976
10. Hartl F, Hayer-Hartl M. Molecular chaperones in the cytosol: from nacent chain to folded protein. Science. 2002; 295:1852-1858
11. Sherman M Y, Goldberg A L. Cellular defenses against unfolded proteins: a cell biologist thinks about neurodegenerative diseases. Neuron 2001; 29(1):15-32.
12. Papp E, Csermely P. Chemical chaperones: Mechanisms of action and potential use. Handb Exp Pharmacol 2006; 172:405-16
13. Nogalska A, D'Agostino C, Engel K. W, Askanas V. Sodium phenylbutyrate reverses lysosomal dysfunction and decreases amyloid-β42 in an in vitro model of inclusion-body myositis, Neurobiol. Dis 2014; 65:93-101
14. Cykowski M D, Powell S Z, Appel J W et al. Phosphorylated TDP-43 (pTDP-43) aggregates in the axial skeletal muscle of patients with sporadic and familial amyotrophic lateral sclerosis. Acta Neuropathol Commun. 2018; 6(1):28
15. Statland J M, Moore D, Wang Y et al. Rasagaline for amyotrophic lateral sclerosis: A randomized, controlled trial. Muscle Nerve. 2019; 59:201-207
16. Wilhite R, Sage J M, Bouzid A, Primavera T, Agbas A. Platelet phosphorylated TDP-43: an exploratory study for a peripheral surrogate biomarker development for Alzheimer's disease. Future Sci OA. 2017; 3:FSO238
17. Sage J M, Hall L, McVey A, Barohn R J et al. Use of capillary electrophoresis immunoassay to search for potential biomarkers of amyotrophic lateral sclerosis in human platelets. J Vis Exp. 2020: 156
18. Jackson C E, Barohn R J, Gronseth G, Pandya S, Herbelin L; Muscle Study Group. Inclusion body myositis functional rating scale: a reliable and valid measure of disease severity. Muscle Nerve. 2008; 37:473-6
19. Jørgensen A N, Aagaard P, Nielsen J L et al. Physical function and muscle strength in sporadic inclusion body myositis. Muscle Nerve. 2017; 56:E50-E58.
20. Lin A Y, Siener C S, Faino A V et al. Optimizing hand-function patient outcome measures for inclusion body myositis. Neuromuscul Disord. 2020; 30;807-814
21. Sangha G, Yao B, Lunn D et al. Longitudinal observational study investigating outcome measures for clinical trials in inclusion body myositis. J Neurol Neurosurg Psychiatry. 2021; jnnp-2020-325141

1. A method for treating inclusion body myositis (IBM) in a subject, comprising:
providing a subject that has IBM;
administering phenylbutyrate (PBA) to the subject in a therapeutically effective amount to provide a treatment for the IBM;
obtaining at least one biological sample from the subject after initiation of the treatment for the IBM;
measuring a biological indicator in the at least one biological sample, wherein the biological indicator is mitochondrial membrane potential; and
monitoring a change in the biological indicator as an indication of effectiveness of the treatment.

2. The method of claim 1, further comprising:
obtaining at least one blood sample as the at least one biological sample;
measuring mitochondrial membrane potential of lymphocytes in the at least one blood sample; and
determining whether the mitochondrial membrane potential of the lymphocytes has changed in response to the PBA.

3. The method of claim 2, further comprising:
at least partially purifying the lymphocytes from the biological sample;
incubating a marker for mitochondrial membrane potential assessment with the lymphocytes in a medium;
removing excess marker from the medium with the lymphocytes; and
measuring the marker retained in the mitochondria of the lymphocytes.

4. The method of claim 3, wherein the marker is a cationic fluorophore.

5. The method of claim 4, wherein the marker accumulates electrophoretically into mitochondria via negative membrane potential.

6. The method of claim 5, wherein the marker includes a reactive chloromethyl group that forms a covalent bond with thiols on proteins and peptides inside of the mitochondria.

7. The method of claim 4, wherein the marker includes a mitochondrial tracker that can be overall cationic with a fluorescent reporter segment and a reactive chloromethyl group.

8. The method of claim 1, further comprising:
obtaining at least one initial biological sample from the subject before initiation of the treatment for the IBM;
measuring the biological indicator in the at least one initial biological sample; and
monitoring a change in the biological indicator from the at least one initial biological sample to the at least one biological sample as an indication of effectiveness of the treatment.

9. The method of claim 1, wherein the administration includes from about 1 gram (gm) to 10 gm of PBA a day.

10. The method of claim 1, wherein the administration is daily for at least one week.

11. The method of claim 1, wherein the administration is for at least one month.

12. The method of claim 1, wherein the administration is for at least three months.

13. The method of claim 1, further comprising:
establishing an initial mitochondrial membrane potential prior to the treatment; and
measuring the mitochondrial membrane potential from a plurality of biological samples taken over a period of time, such as at least one week, at least one month, or at least three months.

14. The method of claim 1, further comprising:
measuring Bcl2 in an RNA sample of the at least one biological sample;
measuring Bax in the RNA sample of the at least one biological sample; and
determining a Bcl2/Bax expression ratio in the RNA sample prepared from lymphocytes of each of the at least one biological sample.

15. The method of claim 1, further comprising:
measuring an apoptosis marker in an initial biological sample obtained prior to the treatment;
measuring the apoptosis marker in the at least one biological sample; and
monitoring changes in the amount of apoptosis marker after initiation of the treatment.

16. The method of claim 15, wherein the apoptosis marker is Annexin binding in lymphocytes of the at least one biological sample.

17. The method of claim 1, further comprising:
measuring oxygen radical antioxidant capacity of an initial biological sample obtained prior to the treatment;
measuring the oxygen radical antioxidant capacity in the at least one biological sample; and
monitoring changes in the oxygen radical antioxidant capacity after the treatment.

18. The method of claim 1, further comprising determining the subject to have IBM.

19. The method of claim 1, further comprising screening the subject prior to the treatment, wherein the screening includes one or more of the following inclusion criteria:
the subject has diagnostic criteria for IBM;
the subject has an age ≥18 years;
a woman subject must be post-menopausal, no menses in >12 months, or status post hysterectomy; or
the subject commits to remain on stable concomitant medications throughout duration of the treatment.

20. The method of claim 1, further comprising screening the subject prior to the treatment, wherein the screening includes one or more of the following exclusion criteria:
presence of any one of the following medical conditions in the subject: chronic infection; chronic renal insufficiency; cancer other than skin cancer less than five years prior; multiple sclerosis or prior episode of central nervous system demyelination; or other chronic serious medical illnesses;
presence of any of the following on routine blood screening of the subject: WBC<3000; Platelets<100,000; hematocrit<30%; BUN>30 mg %; creatine>1.5 mg %; or liver disease with serum albumin<3 G/DL;
women who are pregnant or lactating;
coexistence of other muscular disease in the subject;
drug or alcohol abuse within past three months by the subject;
bleeding disorder in the subject;
liver disease in the subject;
congestive heart failure in the subject; or
hypernatremia in the subject.
